(19) United States
(12) Patent Application Publication
(10) Pub. No.: US 2005/0004767 A1
(43) Pub. Date: Jan. 6, 2005

(54) Method and system for validating changes in medical practice, procedures and product choice

(76) Inventor: Sol F. Green, North Woodmere, NY (US)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/834,380

(22) Filed: Apr. 29, 2004

Related U.S. Application Data
(60) Provisional application No. 60/466,434, filed on Apr. 30, 2003.

(51) Int. Cl. G06F 17/60; C12Q 1/68; G06F 19/00; G01N 33/48; G01N 33/50

(52) U.S. Cl. 702/20; 705/3; 435/6

(57) ABSTRACT

A system and method for assessing the statistical and clinical acceptance of a new medical service or changes in products used by a clinical laboratory or hospital, and providing documentation of validation. The system and method includes an information exchange mechanism, such as an internet, extranet, compact disc (e.g., CD-ROM) or text-based mechanism to establish a systematic approach between a provider and client for collecting, reviewing and providing information to demonstrate the clinical validity of new or modified products. The collected information and user requests are evaluated using tools such as an analytical array to determine required protocols, limits and subsequent tests needed to demonstrate the clinical validity of products, and thereafter, to provide a report to show validation of the change.

### CROSS-REFERENCE TO RELATED APPLICATIONS

This application claims priority under 35 U.S.C. §119(e) from a U.S. Provisional Patent Application of Sol Green, entitled “Method And System For Validating Changes In Medical Practice, Procedures And Product Choice”, Ser. No. 60/466,434, filed Apr. 30, 2003, the entire content of which is incorporated herein by reference.

Related subject matter is also disclosed in a U.S. Patent Application of Scott Pardo et al., entitled “System And Method For Determining Clinical Equivalence Of Test Methods”, Ser. No. 10/096,102, filed on Mar. 13, 2002, and in a U.S. Provisional Patent Application of Patricia Harper et al., entitled “Extranet Service Site And Method For Using Same”, Ser. No. 60/466,439, filed on Apr. 30, 2003, the entire content of each being incorporated herein by reference.

### FIELD OF THE INVENTION

The present invention relates to a system and method that can be used for assessing the statistical and/or clinical acceptance of a new medical service or the changes in products used by a clinical laboratory or hospital, and providing documentation of a subsequent validation. Specifically, the system and method provides an internet, extranet, compact disc, or text based mechanism to establish a systematic approach to determine clinical validity. The mechanism identifies, in addition to other features, protocols, limits and subsequent tests, where required, to demonstrate the clinical validity of new or modified products. Once identified, the mechanism can further provide components to help make the desired assessments.

### BACKGROUND OF THE INVENTION

Currently, there is a burdensome challenge placed on laboratories to show clinical and sometimes statistical equivalence for a change in products or procedures in clinical use. When a change is identified, validation demonstrating that the change does not negatively impact clinical practice is typically required. Such validation can often be satisfied with information collected in journals and clinical documentation (i.e. white papers) showing equivalence. However, further testing is often necessary to fill the remaining clinical data gaps that can occur. Such testing can be cost prohibitive at some laboratory locations, and yet performed redundantly throughout many other laboratories.

Such testing of various technologies in the clinical arena has improved in sophistication since manual cell counts and boiled glucose analyses. A growth in instrument and reagent manufacturers has accompanied this improvement in technology leading to an exponential increase in methods available to the laboratorian. As a result, this has led to an expansion of the test methodologies for many products. However, because of this proliferation of methodologies, it is often impossible for a manufacturer to test every instrument/reagent combination on the market with the manufacturer's products, and new technologies are constantly adding new and improved methods and assays.

Regardless of the complications noted above, as manufacturers enhance existing products or develop new products, the manufacturer must demonstrate the safety and efficacy of each change. Clinical testing is crucial to the validation of these products by the manufacturer and during conversions by a laboratory. However, there can be more than 2,000 chemistry analytes alone, and numerous preanalytical and postanalytical systems, making it impractical for a laboratory to test all permutations.

Accordingly, a need exists for a device and method for collecting, reviewing and providing validation information, including protocols, limits and subsequent tests, where required, to demonstrate the clinical validity of new or modified products.

### SUMMARY OF THE INVENTION

It is therefore an object of the present invention to provide a system and method for establishing a systematic approach to demonstrate the clinical validity of new or modified products through an exchange of validation information with a user.

It is another object of the present invention to provide a system and method for establishing a systematic approach to determine generic protocols, clinical limits and subsequent tests, where required, to demonstrate the clinical validity of new or modified products.

It is another object of the present invention to provide a system and method for establishing a systematic approach to determine specific validation steps, such as specifying and documenting which analytes to test to demonstrate clinical validity for new or modified products, for example, glass and plastic tubes.

It is another object of the present invention to provide a system and method for establishing a systematic approach to determine how analytes should be tested or assessed.

It is another object of the present invention to provide a system and method for establishing a systematic approach to supply components to achieve validation, such as those required for analyte tests or assessments.

It is yet another object of the present invention to provide a system and method for establishing a systematic approach to assess the statistical and clinical acceptance or validation of a new medical service or change in products used by a customer, such as a clinical laboratory or hospital.

It is still another object of the present invention to provide a system and method for establishing a systematic approach to provide documentation of the validation and the statistical and clinical acceptance of new medical services or changes in products.

These and other objects are substantially achieved by providing a system and method for an information exchange mechanism, such as an internet, extranet, compact disc or text based mechanism for collecting, reviewing and providing information to demonstrate the clinical validity of new or modified products. The collected information and user requests are evaluated, depending upon what is to be validated, to determine required protocols, limits and subsequent tests needed to demonstrate the clinical validity of products.

The system and method includes a medium for exchanges between a client and a provider, including at least one of an internet web page, extranet web page, compact disc and text-based communication tool for receiving a request from a client for a need to validate a change in a clinical practice or product, wherein the change can require a showing of validated results. The system and method then provides a reply from the provider, including an analytical array mechanism to validate the proposed change, and the client requests at least one element of the analytical array based upon a review of said analytical array. The provider can then research at least one database of existing validation information to determine relevant information and existing information gaps, and provide a testing protocol to validate the change.

### BRIEF DESCRIPTION OF THE DRAWINGS

The above and other objects and advantages will be apparent upon consideration of the following drawings and detailed description. The preferred embodiments of the present invention are illustrated in the appended drawings, in which:

FIG. 1 is a flowchart illustrating an information exchange to determine generic protocols, clinical limits and subsequent tests to demonstrate the clinical validity of new or modified products in accordance with an embodiment of the present invention;

FIGS. 2A-2F are tables illustrating examples of an analytical array spreadsheet for variable consideration in accordance with the exchange mechanism of FIG. 1;

FIG. 3 is a table illustrating an example of a clinical data summary table in accordance with the exchange mechanism of FIG. 1; and

FIG. 4 is a table illustrating an example of a gap analysis table in accordance with the exchange mechanism of FIG. 1.

In the drawing figures, it will be understood that like numerals refer to like elements.

### DETAILED DESCRIPTION OF THE PREFERRED EMBODIMENTS

The embodiments of the present invention described below provide a mechanism to establish a systematic approach to demonstrate the clinical validity of new or modified products through an exchange of validation information with a user. The system and method includes an information exchange mechanism, such as an internet, extranet, compact disc or text based mechanism for collecting, reviewing and providing information to demonstrate the clinical validity of new or modified products. User requests are evaluated, in light of the collected information, to determine required protocols, limits and subsequent tests needed to demonstrate the clinical validity of products. In a specific example described below, the embodiments of the present invention are provided in an application related to tube products (i.e., glass and plastic tube development and/or conversions); however, the invention is applicable to any number of different products or services, and to other fields of use, in the same or similar manner.

In the example described below, the embodiments of the present invention provide a mechanism to determine protocols, limits and tests needed to demonstrate clinical validity. Once a need for tests is determined, the embodiments further serve to determine specific tests, such as which analytes to test, that will demonstrate clinical validity for new or modified products. The mechanism can further be used to determine how the analytes should be tested or assessed, and can provide a feature for supplying specific components required to achieve the assessment. In doing so, the embodiments of the present invention can be used for assessing the statistical or clinical acceptance or validation of new medical services or changes in products used by a customer, such as a clinical laboratory or hospital. Finally, the mechanism can be used to then provide documentation of the completed validation and update one or more databases for use in future validations.

The purpose of the analytical array technique used in the disclosed embodiments of the present invention is to establish a systematic approach to determine specific tests, such as which analytes to test, that will demonstrate clinical validity of new products or systems. As noted above, there is a burdensome challenge placed on laboratories to show a required degree of clinical and statistical equivalence for a change to be allowed in a product or process in clinical use. When a change is identified, a validation procedure demonstrating that the change does not negatively impact clinical practice is typically required. This validation can be satisfied in a number of ways, such as with information collected from journals and clinical documentation (i.e. white papers) showing equivalence.

Typically however, further testing is necessary to fill the remaining clinical data gaps or validation specifics, which are unavailable in existing papers. Testing to fill such gaps can be cost prohibitive in many cases, and can often be performed redundantly throughout many unrelated laboratories. Therefore, the systematic approach using an analytical array technique described in greater detail below can be used to reduce or eliminate the clinical documentation gaps by establishing communication with a client, identifying and implementing a validation procedure, and thereafter documenting collected test results. Such a technique avoids the need to perform tests where previous documentation can demonstrate that there is no longer a need to perform that specific test again.

Embodiments of the present invention also provide a mechanism for converting collected clinical testing results into a presentable report generated for showing clinical efficacy of a new or modified medical product or change in use. These, and other objects are achieved by providing an internet, extranet, compact disc or text based mechanism to execute a sequence of steps which, when performed, yield a displayable report that shows clinical efficacy in establishing the change.

As known to those skilled in the art, communication mediums such as internet, extranet, compact disc or text based mechanisms, can include existing hardware and software to couple users for exchanges, and typically follows a conventional client-server model. The terms “client” and “server” are used to refer to a computer's general role as a requester of data (i.e. the client) or provider of data (i.e. the server). Within a typical Web environment, Web browsers reside in clients and Web documents reside in servers, and the clients and servers communicate using a protocol called “HyperText Transfer Protocol” (HTTP). Locations within the Web environment are defined as sites, and each typically includes a standardized uniform resource locator (URL) that identifies the site. A browser is used to open a connection to a server, or site, and initiate a request for a document. The server delivers the requested document, typically in the form of a text document coded in a standard Hypertext Markup Language (HTML) format. When the connection is closed in the above interaction, the server serves a passive role, i.e., it accepts commands from the client and cannot request the client to perform any action. In still another version, a text based medium can be provided, which serves the same functions noted above, but not requiring the noted hardware and software.

The embodiments of the present invention described below provide a systematic approach using a computer network system (for internet, extranet or compact disc embodiments) or a text system (for text embodiments), where a vendor, manufacturer or outside party directly, or indirectly, assists a client such as a clinical laboratory, hospital or other entity that is required to show that clinical testing has been performed or is not required. Such a showing thereby assures that the laboratory is in compliance with all good clinical laboratory guidelines and regulations. Such assurances are typically reviewed by auditing bodies such as the International Organization for Standardization (ISO), Food and Drug Administration (FDA), Clinical Laboratory Improvement Amendment (CLIA), National Committee for Clinical Laboratory Standards (NCCLS), College of American Pathologists (CAP) and so on, upon inspection of a clinical laboratory.

The disclosed embodiments of the present invention serve to convert collected clinical testing results into a presentable report generated for use in showing clinical efficacy by providing a sequence of steps which yield a displayable report. The steps include an interaction and information exchange between a customer, such as a laboratory in the example described below, and at least one outside party, such as a product manufacturer. This exchange can be executed via internet or extranet communications, compact disc (e.g., CD-ROM), text and the like.

For example, the exchanges and work products described below, can be provided via internet or extranet, or as a text based communications. Such a text based communication can include letters, memorandums and/or tables, which can be continuously updated by either party, and maintained at a laboratory for future uses. Collections of text can be arranged and provided as binders, including each element described below and substantially identical to the elements as provided via internet or extranet communication.

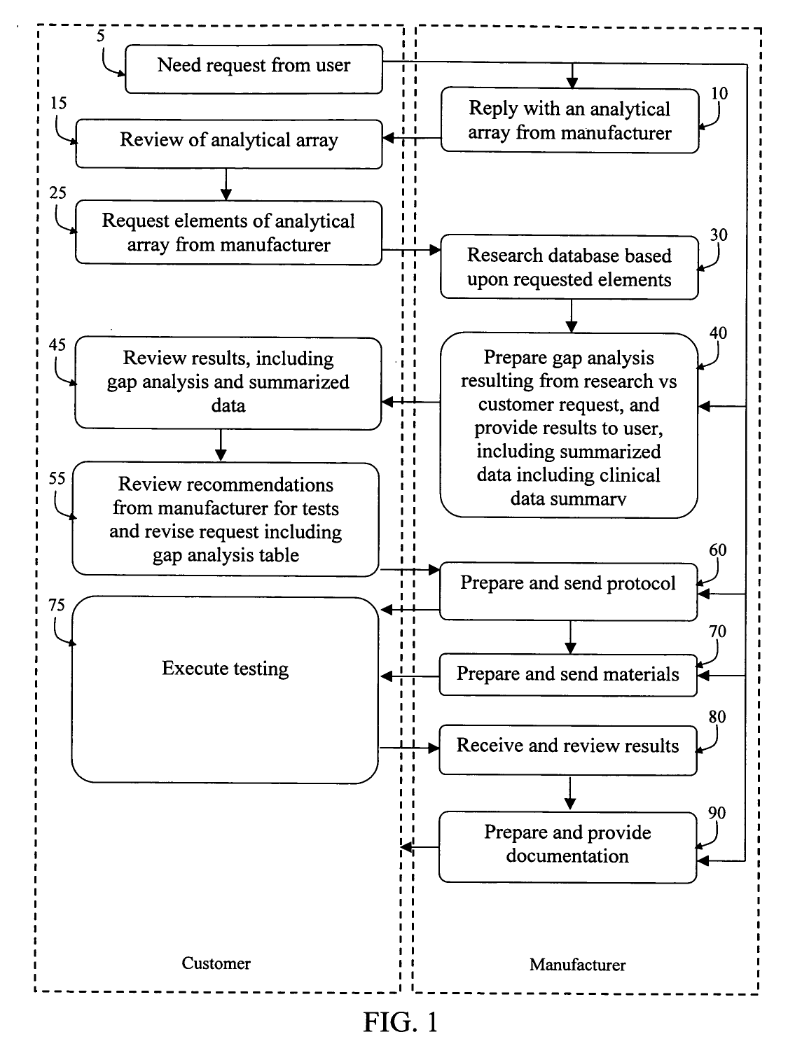

As shown in the flowchart of FIG. 1, the first step 5 consists of a customer or user request communication, such as a laboratory communicating to a manufacturer a need to validate a change in clinical practice at the laboratory, wherein the change might require the showing of validated results. Examples of such a change can include glass to plastic tube conversions, serum to plasma tube conversions, mechanical separator to gel separator conversions, gel separator to mechanical separator conversions, new machines or analyzer changes, reagent supplier changes and the like.

This can be followed by a second step 10 that consists of a reply communication from the manufacturer, wherein the manufacturer presents an analytical array to the laboratory in order to validate the proposed changes in clinical practice. The laboratory can then, in a third step 15, review the analytical array to identify which tests within the array are applicable to the current laboratory needs. In a fourth step 25, the laboratory can then identify and request specific tests within the analytical array from the manufacturer. The tests within the array can be identified and provided to the laboratory in any number of ways, such as via an internet web page survey, having a feature to visually select from among displayed text (i.e., highlighting selected text), as known to those skilled in the art.

In a fifth step 30, the manufacturer can then research at least one database comprising information that teaches what known comparisons have been previously identified in order to reduce the degree of testing required by the current client to show a validated change. The databases can include information assembled from the manufacturer's white papers, literature searches and information known or accessible by the manufacturer. In a sixth step 40, the manufacturer can compare the analytical array tests chosen by the client with the database research to identify which requests can be satisfied with existing information, and remaining information gaps which require testing. The manufacturer can then communicate the comparison results, relevant existing information and required tests to the laboratory for consideration.

In a seventh step 45, the laboratory can interpret the gaps as to what should be tested, including the comparison results, relevant existing information, required tests and clinical ranges recommended by the manufacturer. The laboratory can then review the tests and clinical ranges recommended by the manufacturer in an eighth step 55 in order to confirm or adjust the tests and clinical ranges desired by the laboratory. The laboratory can then communicate a revised request to the manufacturer. The manufacturer can then generate and provide a testing protocol based on the above to the laboratory in a ninth step 60.

The manufacturer can still further provide materials to the laboratory, such as pre-labeled tube sets, control or evaluation samples, or any materials needed for evaluation in a tenth step 70. The laboratory can then use the materials for testing toward the protocol in an eleventh step 75. After testing, the manufacturer can receive and analyze data and test results generated by the laboratory in a twelfth step 80 using an equivalence software package or any number of statistical analysis tools, and thereafter provide to the laboratory, a presentable report that shows validation of the change in a thirteenth step 90. The manufacturer can then update databases to include the new results.

The steps described above can have many alternatives, variations and additional elements. For example, a laboratory may not require the completion of all steps to achieve the desired result. A request for documentation, or validation of a specific change, may only require a limited implementation of the above exchanges (i.e., a customer request for clinical ranges as shown in step 55). A request for documentation from a manufacturer can be completed without the test gap analysis performed above, but can, however, still be achieved through embodiments of the present invention. In the flowchart of FIG. 1, each step can be separately performed without reliance on earlier, or subsequent, steps in other applications of the embodiments of the present invention. Still further variations to the above embodiments can include steps for adding or subtracting results from a portion of the test to the manufacturer's white papers or literature for use in subsequent validation and for other laboratories requiring validation.

In the embodiments of the present invention described above, an outside party or manufacturer can work with a client such as a hospital, clinic or reference laboratory, by storing databases of continuously updated information that can be used to show why testing, or further testing, would not be necessary in some cases. Such databases can be stored within a clinical array to perform future validation gap analysis.

As noted above, a gap analysis is provided in steps five and six, 30 and 40, respectively, wherein the manufacturer determines what known comparisons have been previously identified using a number of tools and databases. In the sixth step 40, the manufacturer compares the analytical array tests chosen by the client with the research to identify which user requests can be satisfied with existing information, and remaining information gaps which require laboratory testing.

The laboratory conversion tools and databases that can be provided by the manufacturer to help perform each step, including the gap analysis, can include the following:
1) Education and technical support performed by a manufacturer or an outside party;
2) Other analytical array testing strategies presented by a manufacturer or an outside party based on the needs of the laboratory;
3) Current white papers provided by a manufacturer or an outside party to reduce future gaps found in the identification stage;
4) Literature searches kept in databases by a manufacturer or an outside party;
5) Clinical limits recommended by a manufacturer or an outside party and agreed upon by the laboratory;
6) Clinical data summary tables and analytic array spreadsheets generated by a manufacturer or an outside party and continuously updated with results of new studies;
7) Generic protocols generated by a manufacturer or an outside party, preferably automatically;
8) Prepared validation supplies, such as tubes, provided by a manufacturer, outside party or a third party;
9) Equivalence software or any statistical analysis tool, preferably provided by a manufacturer or an outside party; and
10) Instrument/reagent/tube results data bases kept by a manufacturer or an outside party and updated by new studies.

The above tools can be combined as part of the comprehensive analytical array testing mechanism in accordance with an embodiment of the present invention. As noted earlier, new technologies are constantly adding new methods and assays, and the analytical array testing technique provides a scientific approach to evaluating multiple products and to accommodate a customer's need for clinical performance information in this dynamic environment. In a preferred embodiment illustrated by the example below, the analytical array testing technique is applied to verify performance in converting from one tube type product to another.

Specifically, the analytical array mechanism incorporates a technique which includes providing an information array to a user and containing information on available technologies. A large number of such analytical arrays can be constructed and provided, however, in the example below, an array is discussed which includes the principle of the reaction, mechanism of detection, and instruments and reagents used by specific manufacturers for a series of analytes for use in a tube conversion validation. In the following example, where the analytical array is used to verify performance in converting from one tube type to another, this information further links clinical reports documenting evaluations of specific tubes with specific test methodologies.

When a large number of variables exist and it is impractical to test all, a clinically sound strategy is required for validation. The purpose of the analytical array technique is to establish a systematic approach to analyze a large number of variables, and in this example, to determine specific analytes to test that will demonstrate clinical validity of an outside party's products to a client, such as a laboratory. As an outside party, or manufacturer enhances existing products or develops new products, they must demonstrate the safety and efficacy of each. Clinical testing is crucial to the validation of these products by the manufacturer and during a conversion by the laboratory. In many cases it is impractical to test all permutations, however, an array concept can be used to successfully review a large element of the clinical performance of products. This system relies on testing a spectrum of analytes that represent all appropriate areas of consideration, as indicated on a spreadsheet and described in greater detail below.

In a first application example, embodiments of the present invention can be applied to the institutional conversion from one product to an alternate product, such as currently available blood collection tubes. Glass evacuated tubes for blood collection have been sold for over 40 years, and one such example includes glass serum separator tubes (i.e., SST). The SST tubes typically contain a thixotropic gel barrier between the serum and the clot, and create a closed system for collecting, transporting, separating and processing blood in a closed tube. However, plastic tubes have recently been made available with documented analytic equivalency to glass tubes for some routine chemistry tests and hematology tests, and provide less risks from breakage than those associated with glass tubes.

In the first application example, embodiments of the present invention are used to help facilitate an institutional change from one tube type to another, i.e. converting from the glass to plastic tubes described above. Embodiments of the present invention allow a manufacturer of plastic tubes to help facilitate such a change using the above steps, wherein the steps provide the following tools via a comprehensive analytical array mechanism, typically following a preanalytical phase. A summary of the main tools include:
1) a review of relevant manufacturer white papers;
2) a literature search;
4) a review of manufacturer or outside parties' information, such as clinical data summary tables;
5) a gap analysis using the laboratory conversion tools (i.e., gap analysis tools) noted above; and
6) an evaluation of statistical and clinical differences as well as potential interference.
   These tools, implemented through the steps of FIG. 1 outlined above, can determine what analytical testing, if any, may be necessary for the customer to convert successfully from one tube type to another.

A preanalytical phase should be considered during implementation, and starts from the time a customer, such as a physician, orders a test, and extends to the time the specimen reaches the test or analyzer. Many preanalytical elements can influence specimen integrity and ultimately the analytical result. Therefore, it is essential to consider all preanalytical variables, including collection variables (i.e. mixing of the specimen, clot times, spin times, serum vs. plasma, specimen transport time and temperature and volume), processing variables (i.e., centrifuge speed, time and temperature), and storage variables (i.e., time and temperature), to assure that clinical comparisons among tube types reflect only tube differences and do not reflect changes in other preanalytical variables as well.

For example, with the proliferation of safety-engineered products (i.e., breakage resistant plastic tubes), product training is important for proper use and protection. Attention must be given to safety device usage, needle disposal and patient care, and these tasks can distract the collector's attention away from proper specimen handling techniques. In yet another example, correct mixing is a critical step for proper product performance of plastic tubes. Laboratories that switch from glass to plastic tubes will reduce glass breakage issues, but can see an increase in preanalytical errors without appropriate handling. Therefore, training in the use of safety-engineered devices creates an opportunity to train and reinforce specimen handling techniques to minimize adverse preanalytical effects.

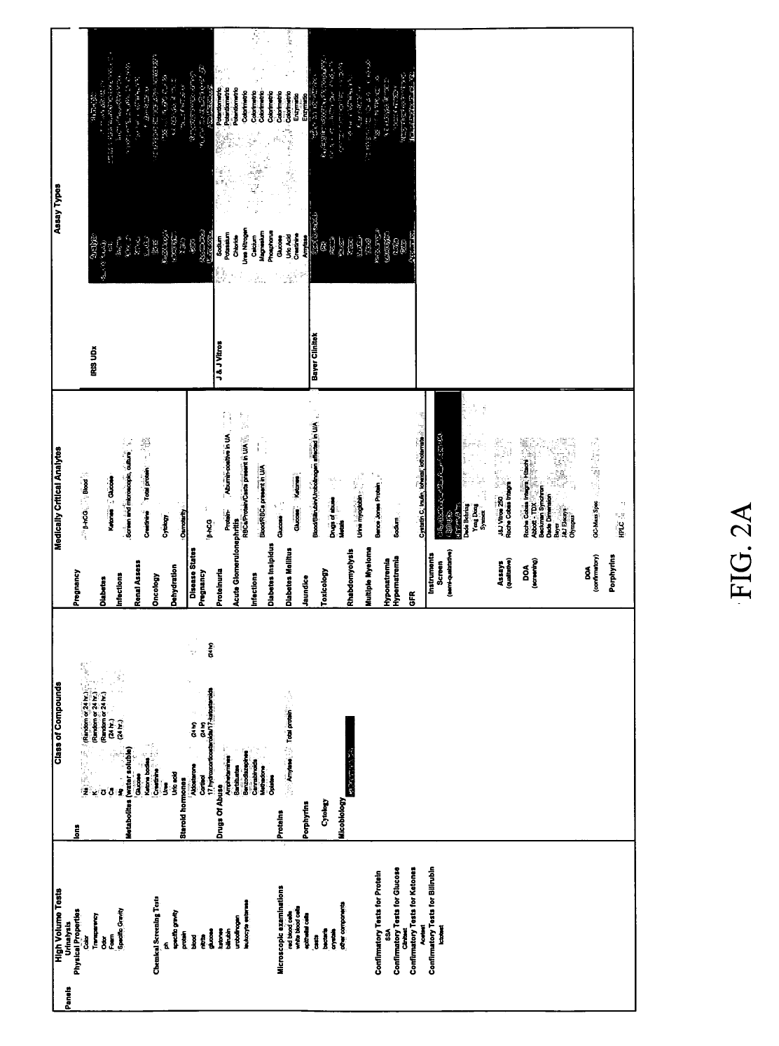

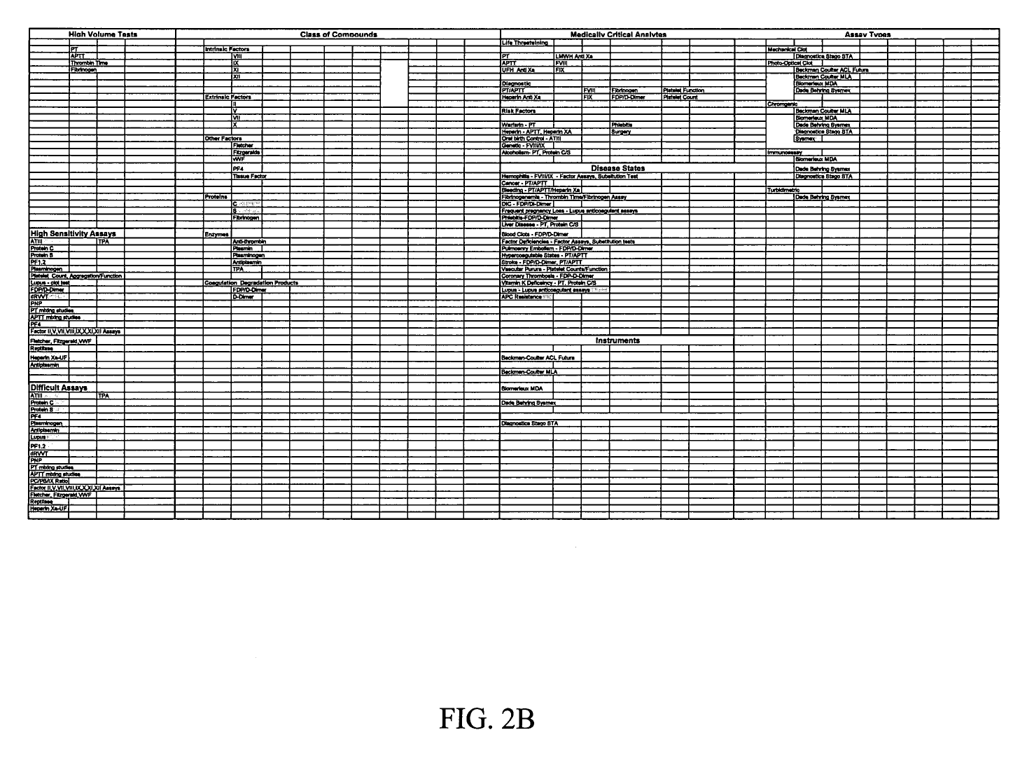

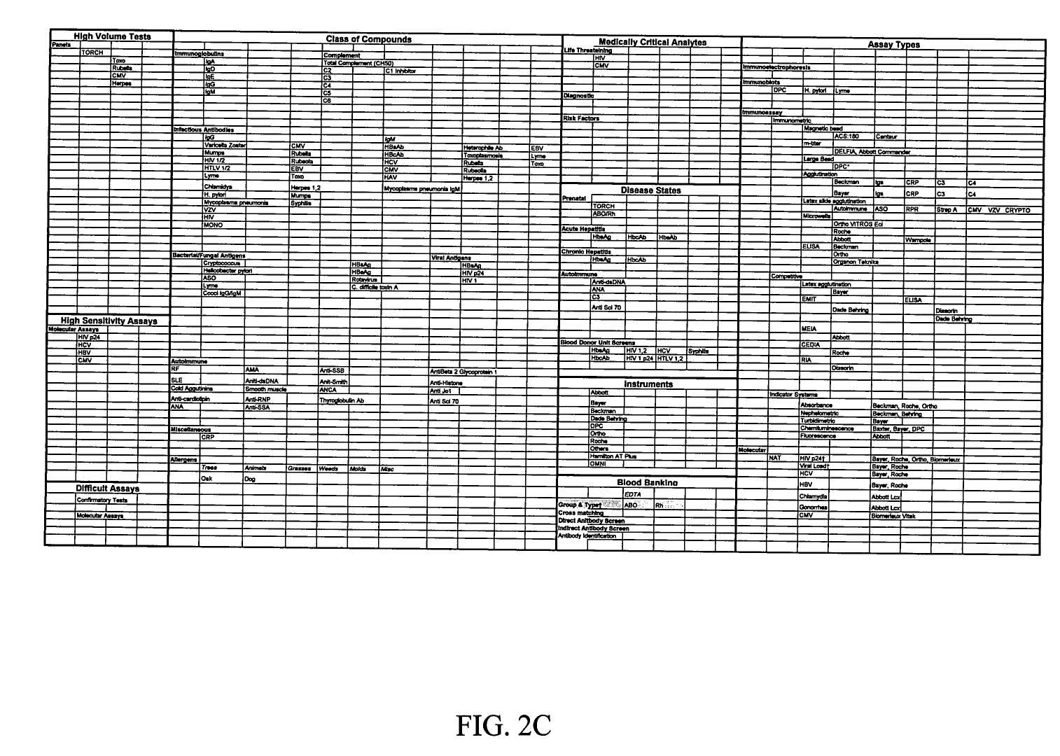

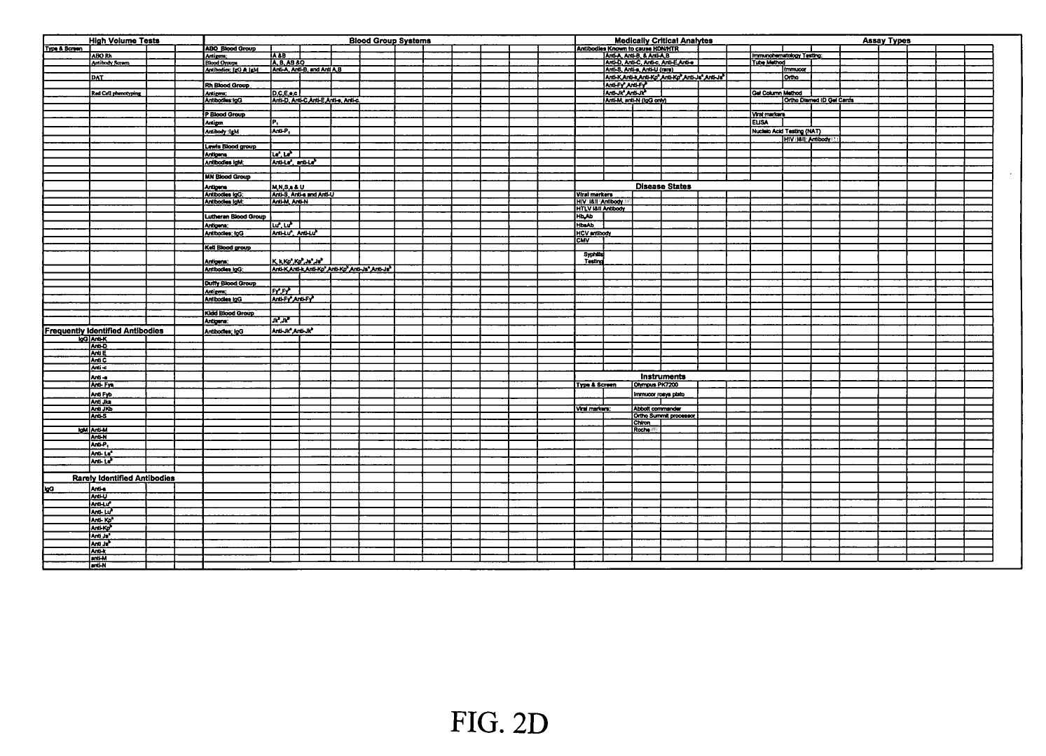

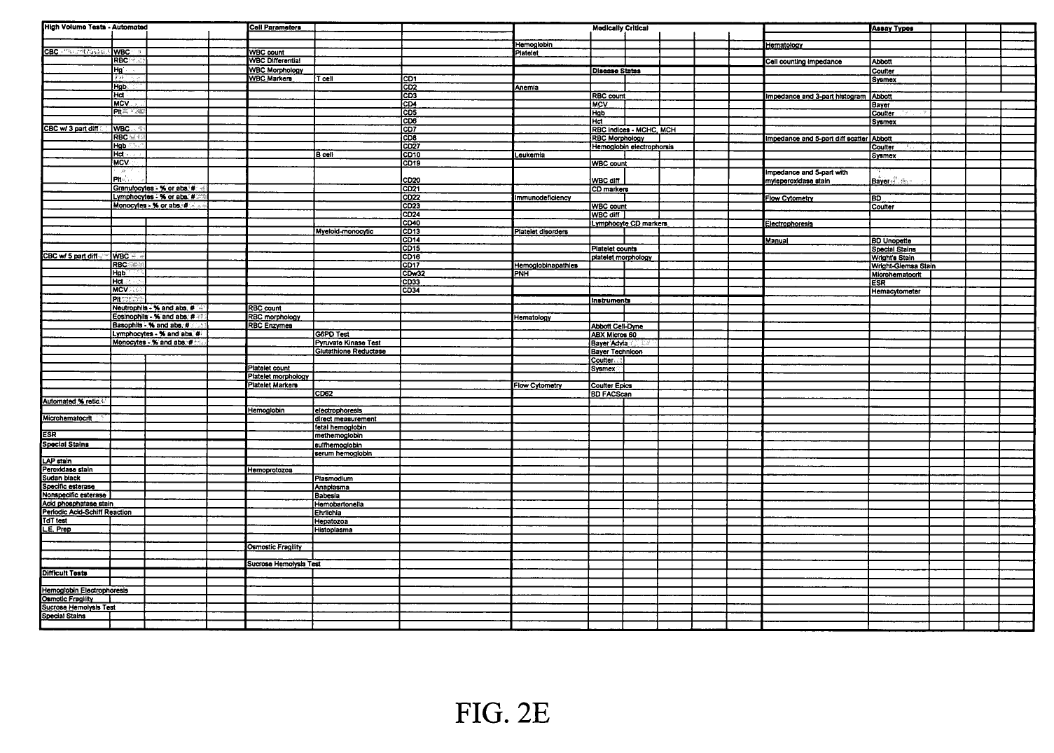

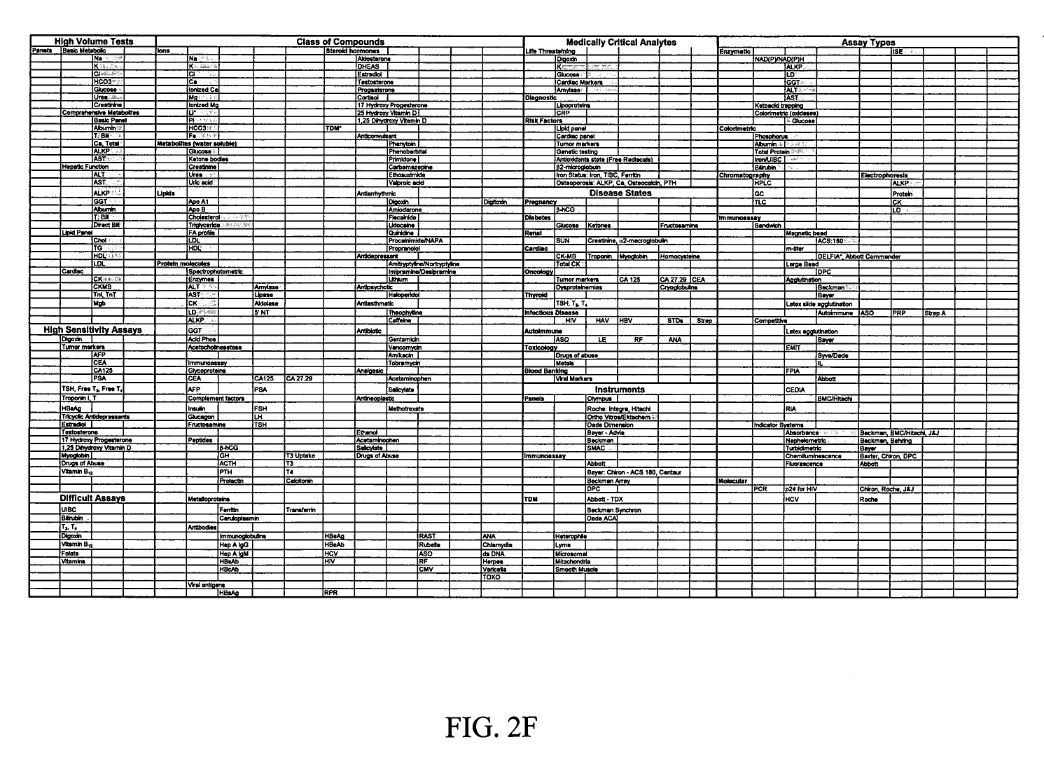

In FIGS. 2A through 2F, tables illustrating examples of an analytical array spreadsheet for variable consideration are shown as provided in the flowchart of FIG. 1 in accordance with an embodiment of the present invention. Typically, a laboratory that wishes to convert from glass to plastic serum tubes can test serum analytes that the laboratory tests most often (i.e. high volume), serum analytes that require the greatest sensitivity, serum analytes that span the range of different classes of compounds (i.e. hydrophobic to hydrophilic, organic and inorganic compounds, and the like), and serum analytes that are the most medically significant. The selected analytes can be tested by a variety of methods on different instruments most commonly found in the laboratory.

The inclusion of different patient populations is also necessary to cover a wide range of values. Following the successful testing of specific analytes in these classifications, the laboratory can then demonstrate that the plastic tube is valid for most analytes. Given the array tables, such as those of FIGS. 2A through 2F, the laboratory can cover the different categories by selecting representative analytes, which if chosen correctly, will greatly reduce the amount of testing required to validate a product, without compromising quality in the laboratory's environment.

As noted above in FIG. 1, a first step 5 consists of a request communication to a manufacturer for a need to validate a change in clinical practice at the laboratory, followed by a second step 10 that consists of a reply communication, wherein a manufacturer presents one or more analytical array tables, such as the array tables shown in FIGS. 2A through 2F, to the laboratory in order to validate the proposed changes in clinical practice.

The tables of FIGS. 2A through 2F, illustrate examples of an analytical array testing technique and clinical documentation in accordance with an embodiment of the present invention. The primary areas for test selection are shown in different boxes in one or more tables, and can include high volume tests, highly sensitive assays, difficult assays, class of compound or analyte, medically critical analytes, disease states, instruments, blood banking, and assay types, in addition to any number of related or additionally required categories. Within these categories, subcategories (i.e. secondary levels) and individual analytes are listed as examples.

In this example, the high volume test category can be broken down into panels and specific tests. The major chemistry panels tested by a chemistry department consist of basic metabolic tests, comprehensive metabolic tests, lipid profiles, and hepatic function testing. These include other specific tests that are high volume that are not included in the panels, e.g. R-HCG (pregnancy), TSH and some drugs.

The analytes listed in the high sensitivity assays category are very responsive to changes in an individual's metabolic status. A small change in the analyte's concentration can result in a large shift in the patient's condition. Some of these analytes are present in the patient's blood at very small concentrations, relative to other analytes.

Difficult assays category includes assays that are sensitive to contamination from compounds similar in structure and/or chemistry to the analyte of interest (i.e. bilirubin). There are also assays that may be difficult to perform (i.e. vitamins) and require some sample preparation before analysis (i.e. folate, UIBC, cyclosporine).

In the class of compound category, several types of compounds are present in serum/plasma. These include: ions/metals (i.e. sodium, potassium, calcium, etc), lipids (i.e. cholesterol, triglycerides, lipoproteins), TDM (i.e. therapeutic drugs), toxicology (i.e. drugs of abuse), metabolites (i.e. glucose, creatinine, uric acid), proteins (i.e. total protein, albumin, some hormones) and steroids (i.e. testosterone, estradiol, progesterone). Each of these classes of compounds has specific chemical characteristics, and the chemical interactions of each of these may differ depending on the types of components that are put in the tubes.

The medically critical analytes category indicates a group in which changes in the analyte concentration of these analytes could be life threatening for the patient. These analytes are also critical for diagnosing specific conditions or used to determine a patient's risk for a particular disease, and a false result would be detrimental to the patient's health. The disease states category represents some specific disease conditions that are grouped into pregnancy, renal, thyroid, infectious disease, transfusion medicine, diabetes, cardiac, oncology, autoimmune, and toxicology categories. As noted above, it is important to include different patient populations in the evaluations. These patient samples provide specific analyte values across a wide range and allow the embodiments of the present invention to test different types of analytes. Patient specimens are also important in ruling out potential interference for qualitative assays (i.e. HbsAg).

An instrument category can include systems that are grouped into three types for chemistry testing, including those types primarily performing panels, immunoassays, or TDMs. However, instrument manufacturers are consolidating these different platforms onto one system to improve laboratory efficiency, and within this category, each of the major manufacturers provide a variety of assays. The top panel systems currently are Roche Integra® or Hitachi®; Dade Dimension® RxL; Bayer Advia®, Ortho Clinical Diagnostics, Inc., and Vitros™. The major immunoassay systems currently include Abbott, Bayer/Chiron, Beckman and Roche. The TDM instruments currently include Abbott, Roche, Dade and Beckman. These are presented as examples, and can be replaced or supplemented by additional manufacturers as required.

The assay types category illustrates types of assays that define the methodology that is used to measure a particular analyte. These are grouped into the following categories: enzymatic, immunoassay, ion specific electrodes (i.e. ISE), colorimetric, chromatographic, electrophoretic. The latter two are highly specialized types of methods and are usually performed in a special chemistry lab. Enzymatic, colorimetric and ISE are methods used to measure the common analytes in the panel profiles. These have been improved over the years so that they are all fairly robust. Enzymatic assays are used to measure the activity of many blood enzymes, such as LDH, ALKP and AST.

Colorimetric assays are also known as photometric or spectrophotometric. The analyte of interest reacts with a reagent substrate to induce a color change in the reaction mixture, and the light intensity at a single or multiple wavelengths is proportional to the concentration of the analyte.

Immunoassays are analytical tests that use antibodies for qualitative or quantitative detection of the compound. There are two important properties of antibodies that characterize immunoassays as a method. These are the unique specificity for the compound to which they bind, and the strength of the binding once formed. The remarkable specificity of antibodies enables minute concentrations of an analyte to be measured in the presence of many closely related substances.

Chromatographic and electrophoretic assays separate the analytes of interest from other similar compounds in a gaseous (i.e. gas chromatography), liquid (i.e. liquid chromatography or capillary electrophoresis) or solid (i.e. electrophoresis) phase. These are complex methods that can require a sample preparation step before analysis.

Molecular assays measure nucleic acids (i.e. DNA or RNA) in blood or plasma. Analyses of these compounds are not generally performed from a serum specimen. Molecular assays are also very specialized methods that are very sensitive to contamination. For example, genetic diseases and viral markers are determined by molecular methods.

The blood banking category is a special category that uses a serum or plasma tube for typing (i.e. Rh) and grouping (i.e. ABO) a patient's blood. These types of samples are also used to screen for cellular antibodies and cross matching blood units for transfusion.

FIGS. 2A through 2F provide an example view of the analytes and methods related to a user validation request, and identify where clinical documentation exists. Specifically, a category can be highlighted or shaded to indicate if documentation in that category currently exists. Additionally, the highlight and shading can be varied by color to indicate in what form the available documentation is provided (i.e., white papers). As noted above, when the user, or laboratory, receives one or more analytical array tables, such as those of FIGS. 2A through 2F, the laboratory identifies which elements within the array are applicable to the laboratory in the third step 15. In a fourth step 25, the laboratory then identifies and requests the specific elements chosen within the analytical array from the manufacturer.

The analytical array tables have categories for the primary areas of consideration in the validation of this product example, and serve as a guideline for any remaining clinical testing required for converting from one tube type to another. However, any number of arrays can be created for validation purposes. The array tables provided in the present example outlined in FIG. 1 are generally applicable to serums, and by selecting items from the primary categories, the laboratory has a better understanding of how the tubes perform, and the clinical performance evaluation can be accomplished in a cost effective and timely manner. The test strategy can be tailored to each conversion to demonstrate clinical validity.

Once requested, the manufacturer can then research at least one database comprising information that teaches what comparisons have been previously identified in order to reduce the degree of testing required by the laboratory to show a validated change in a fifth step 30. The manufacturer can compare the analytical array tests chosen by the laboratory in the fourth step 25 with the database research of the fifth step 30 to identify information gaps which require testing, and communicate the comparison results and required tests to the laboratory for consideration in a sixth step 40. As noted above, the gap analysis uses the laboratory conversion tools including educational and technical support; white papers; literature searches; clinical limits and generic protocols.

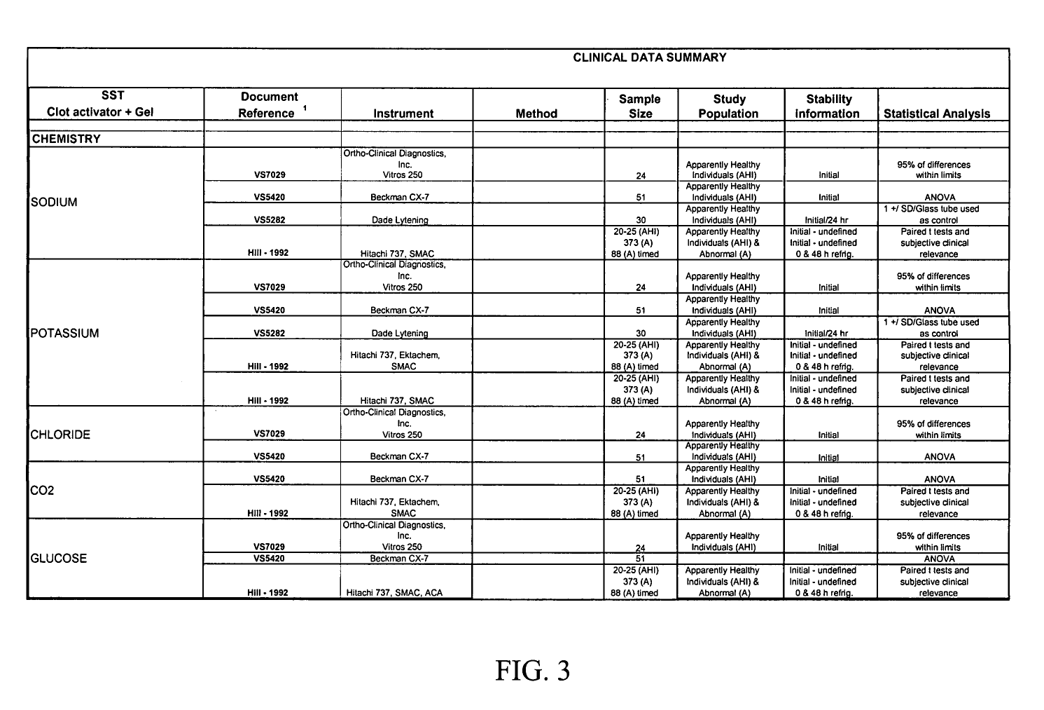

In the case of white papers, the manufacturer or outside party can routinely conduct clinical trials (i.e. internal and external), independently or collaboratively, in order to provide customers with data on products. Most studies can be designed to simulate typical customer usage, and can compare analytical, visual or physical results between different types of tubes. The results of these studies can be distributed to customers in the form of white papers, and relevant white papers can then be summarized into clinical data summary tables, an example of which is shown in FIG. 3. The summary table can include a number of columns and rows indicating available documents associated with chemistries, instruments, sample sizes, and so forth, and can be communicated with the comparison results and required tests to the laboratory in the sixth step 40.

Typically, prior to any tube conversion, the manufacturer recommends a review of all relevant white papers and literature to help assess and/or design clinical studies that can be required before tube conversion. In the case of literary searches, the manufacturer can conduct and provide these literature searches on various tube comparisons. Additionally, since new manuscripts are published monthly in a variety of journals, it can be recommended that each user conduct its own search. The manufacturer can welcome opportunities for collaboration and publications, and where provided, appropriate references for a particular conversion can be added to the summarized data. The summarized data is designed to help assess which analytes can be determined as acceptable based on the white papers, literature, papers relevant to any conversion and periodic updates, and analytical array assessments. The summarized data can also contain summaries of any number of studies (i.e. design, analysis, and outcome).

Following this assessment, an analysis of the gap can be conducted by the laboratory. As noted above, the manufacturer researches the summarized data in order to reduce the degree of testing required by the laboratory to show a validated change in the fifth step 30. The manufacturer can compare the analytical array tests chosen by the user with the database research to identify information gaps which require testing, and communicate the comparison results and required tests to the laboratory for consideration in a sixth step 40. Specifically, the laboratory can then interpret any gaps as to what should be tested, including the comparison results, required tests and clinical ranges recommended by the manufacturer in a seventh step 45. The laboratory can then confirm or adjust the tests and clinical ranges desired by the laboratory, and can then communicate a revised request to the manufacturer in an eighth step 55.

In preparing the revised request of step eight, the laboratory selects the summarized data relevant to its conversion type and reviews the data for relevant white papers and literature against the required test menu. For missing information (i.e. gaps identified by the manufacturer), the manufacturer or outside party can recommend that the laboratory complete appropriate columns (i.e. reference, sample size, study population analytical method, and so on) from its own evaluations and testing.

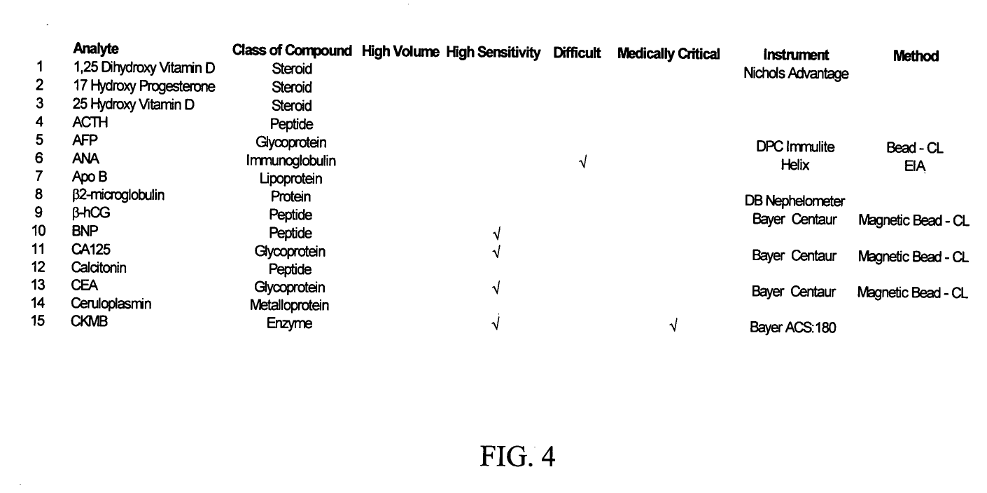

Using the analytical array spreadsheet and the summarized data, the customer is directed in the eighth step 55 to:
1) add information, such as all relevant required analytes into the appropriate categories (i.e. high volume assays, difficult assays, class of compounds, and the like);
2) indicate (i.e., color-code) all analytes that have significant or non-significant differences between control and evaluation samples from the analytical array spreadsheet; and
3) indicate any analyte required by the laboratory and not already documented in the spreadsheet.
   In doing so, the laboratory can identify the remaining analyte gaps, problem analytes or analytes requiring a reference range change to the manufacturer in a gap analysis table. These will be the analytes the laboratory may want to further investigate. FIG. 4 is an example of an analytic array gap analysis table for a glass to plastic SST conversion. As shown in the table of FIG. 4, problem analytes are identified and noted to the manufacturer according to class of compound, volume, sensitivity, instrument and method. Still other designation categories can be provided in other examples.

Once identified, the manufacturer can then generate and provide a testing protocol to the laboratory based on the above in a ninth step 60. The manufacturer can also provide materials to the laboratory, such as pre-labeled tube sets, control or evaluation samples, or any materials needed for the evaluations. The laboratory can then use the materials for testing toward the protocol. After testing, the manufacturer can receive and analyze data and test results generated by the laboratory using an equivalence software package or any number of statistical analysis tools, and thereafter, provide to the laboratory, a presentable report that shows validation of the change.

As noted above, the embodiments of the present invention can also provide a mechanism for converting collected clinical testing results into a presentable report for use in showing clinical efficacy in allowing for a change in a new or modified medical product or change in use. The report can be structured as book indicating all analytes, white papers and the like, and include one or more gap analysis studies which can be combined to define a protocol. In doing so, a single document or book can be provided for complete documentation.

After completing an evaluation in accordance with an embodiment of the present invention, the laboratory statistically analyzes the data. Upon reviewing the data and statistical conclusions, the laboratorian may recognize that some statistical differences may not be clinically different, therefore the laboratorian can apply clinical limits to the data to determine acceptability. Medical usefulness of such a diagnostic test result is an estimate of the magnitude of analytically significant changes in analyte values. Analyte concentrations can result in negative or positive biases, which must fall within acceptance limits small enough for physicians to arrive at a correct diagnostic decision. To be able to give the correct diagnosis, physicians must be able to distinguish if the observed analytical bias is caused by inherent variability of the analytical procedure or by modifications in the patient's physiology and pathology. Therefore, it can be useful to apply clinical limits to the data to determine acceptability.

Embodiments of the present invention can provide a statistical tool to test for equivalence that can analyze many different analytes (i.e., chemistry panel) simultaneously and apply internal clinical limits. The embodiments described above utilize tools such as a comprehensive analytical array testing technique, white papers, literature searches, clinical data summary tables and analytical array spreadsheets, gap analysis, and statistical and clinical differences, as well potential interference. These steps can be used to determine what analytical testing, if any, may be necessary for an institution to convert from one tube type to another.

Whenever changing any manufacturer's blood collection tube type, size or storage condition for a particular laboratory assay, the laboratory personnel can review the tube manufacturer's data and their own data to establish and/or verify the reference range for a specific instrument/reagent system. Based on such information, the laboratory can then decide if the change is appropriate.

For best implementation, the embodiments of the present invention described above can be provided to a customer via an extranet site, internet connection, compact disc or published text, which provides user support in the above form of clinical data regarding rapidly developing products, such as glass and plastic evacuated blood collection tubes for most, if not all, analytes evaluated in the clinical laboratory setting. The data obtained can be used to provide compliance support to clinical laboratory customers or site users that may be developing or converting any number of products beyond those described above.

Although only a few exemplary embodiments of the present invention have been described in detail above, those skilled in the art will readily appreciate that many modifications are possible in the exemplary embodiments without materially departing from the novel teachings and advantages of the invention. Accordingly, all such modifications are intended to be included within the scope of the invention as defined in the appended claims and equivalents thereof.

1. A method for assessing the statistical and/or clinical acceptance of a new medical service or the changes in products used by a client, such as a laboratory or hospital, and providing documentation of a subsequent validation, the method comprising:
providing a medium for exchanges between a client and a provider, said medium including at least one of an internet web page, extranet web page, compact disc and text-based communication tool;
receiving a request from said client via said medium for a need to validate a change in at least one of a clinical practice and product, wherein said change can require a showing of validated results;
providing a reply from said provider via said medium, including an analytical array mechanism to validate said proposed change;
reviewing a request from said client via said medium that identifies at least one element of said analytical array chosen by said client based upon a review of said analytical array by said client; and
researching at least one database comprising information that includes existing validation information and comparing said elements chosen by said client with said existing validation information to determine relevant information and existing information gaps which require testing, and providing said relevant information and existing information gaps which require testing to said client via said medium for review.

2. A method for assessing the statistical and/or clinical acceptance of a new medical service or the changes in products used by a consumer as claimed in claim 1, further comprising:
reviewing a revised request from said client, and preparing and providing a testing protocol to validate said change in at least one of a clinical practice and product to said client via said medium.

3. A method for assessing the statistical and/or clinical acceptance of a new medical service or the changes in products used by a consumer as claimed in claim 1, further comprising:
reviewing a test result from said client, and preparing and providing a documentation of a subsequent validation to said client via said medium.

4. A method for assessing the statistical and/or clinical acceptance of a new medical service or the changes in products used by a consumer as claimed in claim 1, further comprising:
providing said analytical array via said medium and having a logical arrangement of information elements identified by a plurality of icons including at least one of a text icon and a figure icon.

5. A method for assessing the statistical and/or clinical acceptance of a new medical service or the changes in products used by a consumer as claimed in claim 4, wherein said information elements include at least one of a test information element, compound element, instrument element, analyte element and assay type element.

6. A method for assessing the statistical and/or clinical acceptance of a new medical service or the changes in products used by a consumer as claimed in claim 4, wherein said icons are distinguished in said array according to a type of documentation available for said element represented by said icon.

7. A method for assessing the statistical and/or clinical acceptance of a new medical service or the changes in products used by a consumer as claimed in claim 6, wherein said icons are distinguished in said array by at least one of a shaded and a highlighted icon.

8. A method for assessing the statistical and/or clinical acceptance of a new medical service or the changes in products used by a consumer as claimed in claim 6, wherein said documentation includes at least one of an education and technical support performed by said manufacturer, current white papers provided by said manufacturer, literature searches kept by said manufacturer, clinical limits recommended by said manufacturer, clinical data summary tables and analytic array spreadsheets generated by said manufacturer, wherein said documentation is continuously updated.

9. A method for assessing the statistical and/or clinical acceptance of a new medical service or the changes in products used by a consumer as claimed in claim 2, further comprising:
providing materials to said client for performing said testing protocol to validate said change.

10. A method for assessing the statistical and/or clinical acceptance of a new medical service or the changes in products used by a consumer as claimed in claim 9, wherein said materials include at least one of a pre-labeled tube set, control sample, evaluation sample and evaluation material.

11. A method for assessing the statistical and/or clinical acceptance of a new medical service or the changes in products used by a consumer as claimed in claim 3, further comprising:
reviewing said test result from said client using an equivalence software package.

12. A method for assessing the statistical and/or clinical acceptance of a new medical service or the changes in products used by a consumer as claimed in claim 3, further comprising:
reviewing said test result from said client using a statistical analysis tool.

13. A method for assessing the statistical and/or clinical acceptance of a new medical service or the changes in products used by a consumer as claimed in claim 3, further comprising:
updating at least one database based upon said review of said test result from said client and said documentation of said subsequent validation.

14. A method for assessing the statistical and/or clinical acceptance of a new medical service or the changes in products used by a consumer as claimed in claim 1, wherein:
said change in at least one of a clinical practice and product includes at least one of glass to plastic tube conversions, serum to plasma tube conversions, mechanical separator to gel separator conversions, gel separator to mechanical separator conversions, new machine changes, analyzer changes and reagent supplier changes.

15. A method for assessing the statistical and/or clinical acceptance of a new medical service or the changes in products used by a consumer as claimed in claim 1, wherein:
wherein said database includes at least one of an education and technical support performed by said manufacturer, current white papers provided by said manufacturer, literature searches kept by said manufacturer, clinical limits recommended by said manufacturer, clinical data summary tables and analytic array spreadsheets generated by said manufacturer.

16. A system for establishing a systematic approach to provide validation functions between at least one client and a provider, the system comprising:
at least one internet, extranet, compact disc or text based medium for exchange to establish communication between at least one client and a provider, including a request communication from said client for a need to validate a change in clinical practice, wherein said change might require a showing of validated results, and to provide an analytical array mechanism to validate said proposed change; and
at least one research database comprising existing validation information and a comparison mechanism to compare at least one analytical array element chosen by said client with said database research to identify information gaps which require testing and to communicate said information gaps to said client for use in revising said validation request.

17. A system for establishing a systematic approach to provide validation functions between at least one client and a provider as claimed in claim 16, further comprising:
said comparison mechanism further adapted to generate and provide a testing protocol based on a revised request from said client.

18. A system for establishing a systematic approach to provide validation functions between at least one client and a provider as claimed in claim 16, further comprising:
said comparison mechanism further adapted to prepare and provide documentation of a subsequent validation to said client.

19. A system for establishing a systematic approach to provide validation functions between at least one client and a provider as claimed in claim 16, further comprising:
at least one of an equivalence software package and a statistical analysis tool to receive and analyze data and test results generated by said client.

20. A system for establishing a systematic approach to provide validation functions between at least one client and a provider as claimed in claim 16, wherein:
said research database includes at least one of an education and technical support performed by said manufacturer, current white papers provided by said manufacturer, literature searches kept by said manufacturer, clinical limits recommended by said manufacturer, clinical data summary tables and analytic array spreadsheets generated by said manufacturer.

21. A system for establishing a systematic approach to provide validation functions between at least one client and a provider as claimed in claim 16, wherein said analytical array mechanism includes a logical arrangement of information elements identified by a plurality of icons including at least one of a text icon and a figure icon.

22. A system for establishing a systematic approach to provide validation functions between at least one client and a provider as claimed in claim 21, wherein said information elements include at least one of a test information element, compound element, instrument element, analyte element and assay type element.

23. A system for establishing a systematic approach to provide validation functions between at least one client and a provider as claimed in claim 21, wherein said icons are distinguished in said array according to a type of documentation available for said element represented by said icon.

24. A system for establishing a systematic approach to provide validation functions between at least one client and a provider as claimed in claim 23, wherein said icons are distinguished in said array by at least one of a shaded and a highlighted icon.
